(19) United States
(12) Patent Application Publication
(10) Pub. No.: US 2008/0045472 A1
(43) Pub. Date: Feb. 21, 2008

(54) TARGETS FOR HUMAN MICRO RNAS IN AVIAN INFLUENZA VIRUS (H5N1) GENOME

(75) Inventors: Samir Kumar Brahmachari, Delhi (IN); Manoj Hariharan, Delhi (IN); Vinod Scaria, Delhi (IN); Beena Pillai, Delhi (IN)

Correspondence Address: SEED INTELLECTUAL PROPERTY LAW GROUP PLLC, 701 FIFTH AVE, SUITE 5400, SEATTLE, WA, 98104, US

(73) Assignee: Council of Scientific and Industrial Research Bharat Biotech, New Delhi (IN)

(21) Appl. No.: 11/693,611

(22) Filed: Mar. 29, 2007

(30) Foreign Application Priority Data
Mar. 31, 2006 (IN) 0925/DEL/2006

(51) Int. Cl. A61K 31/711; A61P 31/16; C12N 15/11; G01N 33/50; G06G 7/48

(52) U.S. Cl. 514/44; 436/86; 536/23.72; 703/11

(57) ABSTRACT

The present invention relates to targets for Human microRNAs in Avian Influenza Virus (H5N1) Genome and provides specific miRNA targets against H5N1 virus. Existing therapies for Avian flu are of limited use primarily due to genetic re-assortment of the viral genome, generating novel proteins, and thus escaping immune response. In animal models, baculovirus-derived recombinant H5 vaccines were immunogenic and protective, but results in humans were disappointing even when using high doses. Currently, two classes of drugs are available with antiviral activity against influenza viruses: inhibitors of the M2 ion channel, amantadine and rimantadine, and inhibitors of neuraminidase, oseltamivir, and zanamivir. There is paucity of information regarding effectiveness of these drugs in H5N1 infection. These drugs are also well known to have side effects like neurotoxicity. Thus there exists a need to develop alternate therapy for targeting the Avian flu virus (H5N1). The present invention addresses this need in the field.

### CROSS-REFERENCE TO RELATED APPLICATIONS

This application claims the benefit of priority under 35 U.S.C. §119 to Indian Patent Application number 925/DEL/2006, filed Mar. 31, 2006.

### BACKGROUND OF THE INVENTION

1.Field of the Invention

The present invention relates to the targets for human microRNAs in Avian Influenza Virus (H5N1) Genome. The invention particularly provides specific miRNA targets against H5N1 virus.

2.Description of the Related Art

The 186 cases (as of March 2006, WHO report) of Avian influenza caused by H5N1 virus in humans with increasing reports of cases in poultry and migratory birds, has created great concern and panic globally. Influenza virus has the capability to re-assort its genetic material thereby giving rise to novel antigenic proteins which can defy the immune response mechanism. These influenza viruses occur naturally among birds. Wild birds worldwide carry the viruses in their intestines, but are usually not affected by them. However, avian influenza is very contagious among birds and can make some domesticated birds, including chickens, ducks, and turkeys etc. very sick and kill them.

Infected birds shed influenza virus in their saliva, nasal secretions, and feces. Susceptible birds become infected when they have contact with contaminated secretions or excretions or with surfaces that are contaminated with secretions or excretions from infected birds. Domesticated birds may become infected with avian influenza virus through direct contact with infected waterfowl or other infected poultry, or through contact with surfaces (such as dirt or cages and droppings) or materials (such as water or feed) that have been contaminated with the virus.

Infection with avian influenza viruses in domestic poultry causes two main forms of disease that are distinguished by low and high extremes of virulence. The “low pathogenic” form may go undetected and usually causes only mild symptoms (such as ruffled feathers and a drop in egg production). However, the highly pathogenic form spreads more rapidly through flocks of poultry. This form may cause disease that affects multiple internal organs and has a mortality rate that can reach 90-100% often within 48 hours.

During an outbreak of avian influenza among poultry, there is a possible risk to people who have contact with infected birds or surfaces that have been contaminated with secretions or excretions from infected birds. Symptoms of avian influenza in humans have ranged from typical human influenza-like symptoms (e.g., fever, cough, sore throat, and muscle aches) to eye infections, pneumonia, severe respiratory diseases (such as acute respiratory distress), and other severe and life-threatening complications. The symptoms of avian influenza may depend on which virus caused the infection.

The Influenza pandemics of 1957 and 1968 which killed millions of people worldwide were thought to arise due to genetic re-assortment of the Influenza A virus genome. The current scenario is a cause of worry as researchers identify the reason for the current spread of influenza in human to be the result of adaptive mutation, the form that arises from mutations stimulated by stress, allowing adaptation to stress and hence considered to be more virulent and contagious. In humans if the virus infects and remains dormant in lung cells it may express during an immunosuppressed stage of the host.

The existing therapies for Avian flu are of limited use primarily due to genetic re-assortment of the viral genome, generating novel proteins, and thus escaping immune response. In animal models, baculovirus-derived recombinant H5 vaccines were immunogenic and protective, but results in humans were disappointing even with high doses.

Currently, two classes of drugs are available with antiviral activity against influenza viruses: inhibitors of the ion channel activity of the M2 membrane protein, amantadine and rimantadine, and inhibitors of the neuraminidase, oseltamivir, and zanamivir. There is a paucity of information regarding the effectiveness of these drugs in H5N1 infection. These drugs are also well known to have side effects like neurotoxicity. This shows that there exists a need to develop alternate therapy for targeting the Avian flu virus (H5N1). The present invention addresses this need in the field.

RNAi (RNA interference) has been implicated for therapy of certain viral infections for example siRNA which has reached clinical trials. Similarly protein levels of Hepatitis C virus (HCV) and Primate Foamy virus (PFV-1) were shown to be regulated by human miRNAs.

miRNA (micro-RNA) is a form of single-stranded RNA which is typically 20-25 nucleotides long, and is thought to regulate the expression of other genes. miRNAs are RNA gene products which are transcribed from DNA, but are not translated into protein. The DNA sequence that codes for an miRNA gene is longer than the miRNA. This DNA sequence includes the miRNA encoding sequence and an approximate reverse complement. When this DNA sequence is transcribed into a single-stranded RNA molecule, the miRNA sequence and its reverse-complement base pair to form a double stranded RNA hairpin loop; this forms a primary miRNA structure (pri-miRNA) followed by its maturation into miRNAs. The function of miRNAs appears to be in gene regulation by inhibiting protein synthesis.

Embodiments of the Invention

One embodiment of the present invention is to provide a novel therapeutic strategy against avian flu virus using human microRNA which obviates the drawbacks of the above mentioned therapeutics.

Yet another embodiment is to provide the miRNAs complementary to H5N1 genes as a novel therapeutic agent.

Still another embodiment is to provide miRNA mediated inhibition of protein synthesis in Influenza A H5N1.

Yet another embodiment is to design modified miRNA as therapeutic for prevention of Avian Flu in poultry.

Yet another embodiment is to provide a method to inhibit avian flu infection in humans.

Yet another embodiment is to provide a method for detection of predisposition to Bird flu in humans.

Still another embodiment is to provide microRNA as novel universal therapeutics against common cold and other influenza viruses.

Novelty of Invention Embodiments

The present invention embodiments provide a novel strategy to target genes of avian flu virus strain H5N1 by human microRNAs. In other embodiments, the invention also provides for the first time the use of microRNA to inhibit protein synthesis of H5N1 virus. The invention also discloses a process to inactivate or block activity of avian flu virus strain H5N1. Further provided is a method of prediction of miRNA targets in avian flu virus H5N1.

### SUMMARY OF THE INVENTION

The invention relates in certain embodiments to targeting of Avian Influenza H5N1 genes with human microRNAs. The invention in certain embodiments provides specific microRNAs targets against Avian Influenza H5N1strain. MicroRNAs are short RNA molecules which have the ability to repress protein synthesis by binding to messenger RNAs and consequently inhibit the viral activity. In the presently disclosed invention embodiments the applicants have screened the Avian Influenza H5N1 reference genome computationally using human microRNAs for identifying targets for Avian Influenza strain H5N1 activity inhibition.

Accordingly, embodiments of the present invention provide targets for Human microRNA in avian flu virus strain H5N1 genome represented by SEQ ID Nos. 1 and 2

The invention in certain further embodiments provides a method for targeting avian flu virus strain H5N1 genome with human miRNAs which comprises:

(a) downloading and computationally shuffling the whole avian flu virus strain H5N1 genome (see, e.g., Xu et al., 1999 Virol. 261:15) sequence from publicly available databases at URL: http://www.ncbi.nlm.nih.gov/genomes/VIRUSES/viruses.html;

(b) computationally predicting the targets for human microRNAs in the said shuffled avian flu virus strain H5N1 genome sequences and computing the cut off;

(c) computationally deriving consensus predictions for microRNA-target pairs, which have scores greater than (>) the cutoff of step (b) for human microRNAs;

(d) mapping computationally the human microRNA targets in the avian flu virus strain H5N1 genome using the miRNA of SEQ ID NOS. 1 and 2.

In an embodiment to the invention, the software programs used for computational predictions consist of software available in public domain, viz., RNAhybrid, miRanda, DIANA-micro-T and microInspector.

In another embodiment to the invention, publicly available software ShuffleSeq was used for shuffling downloaded H5N1 genome to minimize error due to sequence compositional bias.

The parameters used for prediction of targets comprised of sequence complementarity, minimum free energy of the duplex and continuous seed complementarity towards the 5′ end of the microRNA.

In yet another embodiment to the invention the human microRNAs and their respective targets are identified using the four prediction software miRanda, RNAhybrid, MicroInspector and DianaMicroT

Another aspect of the invention is to provide a method for detection of predisposition to Bird flu in humans wherein low miRNA levels are associated with high risk and high miRNA levels are associated with low risk to avian flu.

Still another aspect of the invention is provision of universal miRNA for targeting human and avian bird flu.

Still another aspect of the invention is for design and use of synthetic oligomers that can act as miRNAs which can repress protein synthesis in H5N1 virus. The ribonucleosides of the oligomer can be modified using strategies like locked Nucleic acid (LNA) or 2′—O-methyl RNA (OMe) resulting in better stability and binding to the target mRNA strand, thus enabling the repression of H5N1 proteins therapeutic in birds against Avian flu virus.

### BRIEF DESCRIPTION OF THE SEVERAL VIEWS OF THE DRAWING(S)

FIG. 1 shows positions of the two microRNA targets on the H5N1/A virus genome.

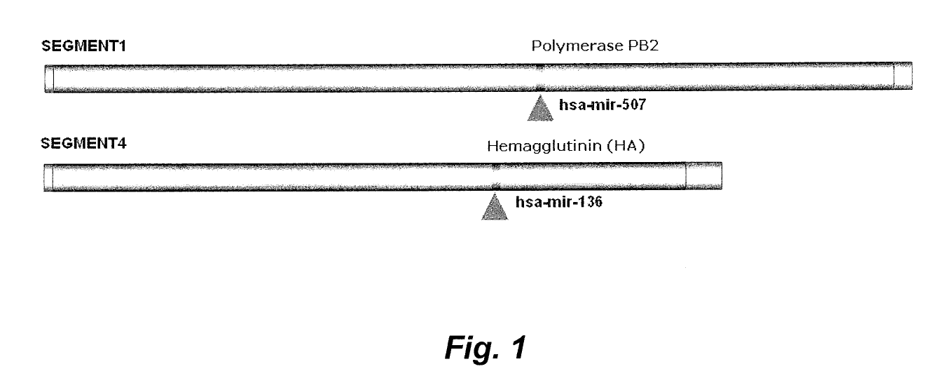

FIG. 2 shows Protocol for Luciferase assay.

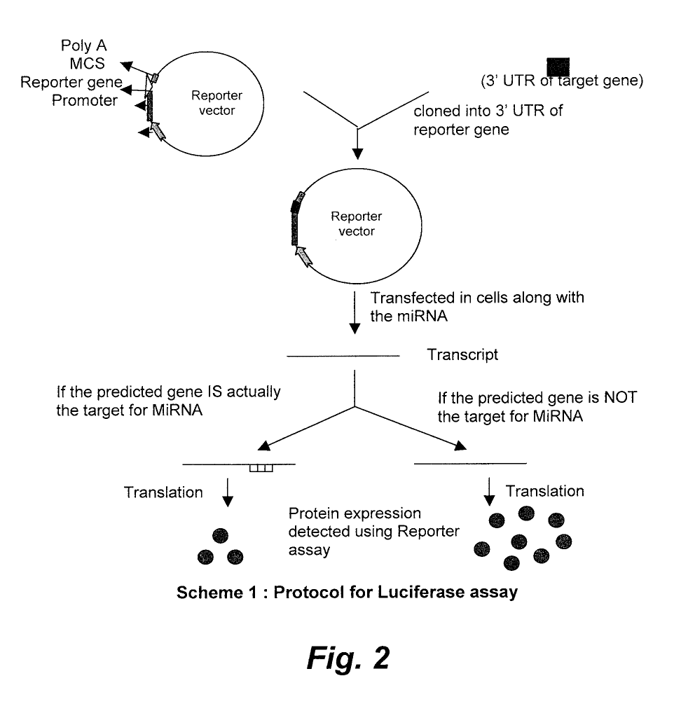

FIG. 3 shows effect of hsa-miR-136 on hemagglutinin gene target.

FIG. 4 shows effect of hsa-miR-507 on PB2 gene.

FIG. 5 shows expression profile of hsa-miR-136 as reported in four lung tissue samples.

FIG. 6 shows miRNA detection in different cell lines using primer extension methods (5 ug RNA and 10 pmol primer), including expression of miR 136 and 507 in neuronal cells and in human alveolar epithelial cell line A549. Lane 1, has-mir-29c (mature sequence-22 bases) in HepG2 cell line; Lane 2, has-mir-29c (mature sequence-22 bases) in A549 cell line; Lane 3, has-mir-29a (mature sequence-23 bases) in A549 cell line; Lane 4, has-mir-507 (mature sequence-23 bases) in A549 cell line; Lane 5, has-mir-136 (mature sequence-25 bases) in A549 cell line; Lane 6, has-mir-29c (mature sequence-22 bases) in Hela cell line; Lane 7, has-mir-29a (mature sequence-23 bases) in Hela cell line; Lane 8, has-mir-507 (mature sequence-23 bases) in Hela cell line; Lane 9, has-mir-136 (mature sequence-25 bases) in Hela cell line.

### DETAILED DESCRIPTION OF THE INVENTION

Data Retrieval

The human microRNA mature sequences were downloaded from miRBase (http://microrna.sanger.ac.uk/). For querying probable targets in the H5N1/A virus genome, we used the RefSeq validated H5N1 reference sequence, obtained from http://ncbi.nlm.nih.gov.

Prediction of miRNA Targets in H5N 1/A Virus

We used four well-established microRNA target prediction softwares miRanda (John et al., 2004 PloS Biol. 2:1862), RNAhybrid (Rehmsmeier et al., 2004 RNA 10:1507), MicroInspector (Rusinov et al., 2005 Nucl. Ac. Res. 33(web server issue):W696) and DIANA-MicroT (Kiriakidou et al., 2004 Genes Dev. 18:1165) to predict targets for the 330 human miRNAs obtained from miRBase in the H5N1 reference sequence. H5N1 targets to human microRNAs were initially predicted using miRanda alone with default parameters (Gap Open Penalty: −8.0; Gap Extend: −2.0; Score Threshold: 50.0; Energy Threshold: −20.0 kcal/mol; Scaling Parameter: 2.0). In order to increase the stringency, a cut-off score was derived above which the miRNA-target pairs were selected. A cut-off score was derived by running the same program on a shuffled sequence of H5N1 reference strain with the same set of miRNAs. H5N1 genome sequence was shuffled using the EMBOSS2 (http://emboss.ch.embnet.org) program ShuffleSeq. This enabled filtering of probable false positive hits and selection of the most probable and high-scoring values. These short-listed H5N1 targets to human microRNAs were also found to be highly probable targets on the other prediction software. Prior to running the RNAhybrid program, the RNA calibrate module was used to derive the xi and theta values for calculation of Extreme Value Distribution. The xi-theta values thus obtained were included as one of the parameters while using RNAhybrid for target prediction. This minimizes the base composition bias. Also, the helix parameters were set to include maximum continuous complementarity towards the 5′ end of the miRNA. It was observed that out of the several probable targets predicted by RNAhybrid, the two filtered pairs from miRanda had the lowest minimum free energy. Similar observations were made when the other two software were employed with default parameters, viz., minimum free energy of −20. The target regions were mapped to the genomes of other H5N1 strains. The target sites for the respective miRNAs were aligned using Clustalw software (e.g., Lopes, 2005 Conf. Proc. IEEE Engineer. Med. Biol. Soc. 3:2843).

Validation of miRNA Targets in H5N1/A Genome

The target of miRNA 136 and 507 found in the Hemagglutinin and PolymeraseB genes of H5N1 genome was further validated by experimental means. The validation was carried out in a cell culture model employing HeLa cells. Primer extension based methods described below were used to ensure that HeLa cells express the miRNAs being tested. A vector with the firefly luciferase gene under the control of a constitutive promoter was used to monitor the activity of the miRNA. Cultured HeLa cells were transfected with various constructs bearing reporter gene which carried testable target regions in their 3′Untranslated regions. Subsequently, the reporter gene activity was monitored using enzymatic assays. The expression level of the reporter would be expected to get downregulated if the cellular miRNA binds to the 3′UTR and results in translational block of the target gene (scheme 1).

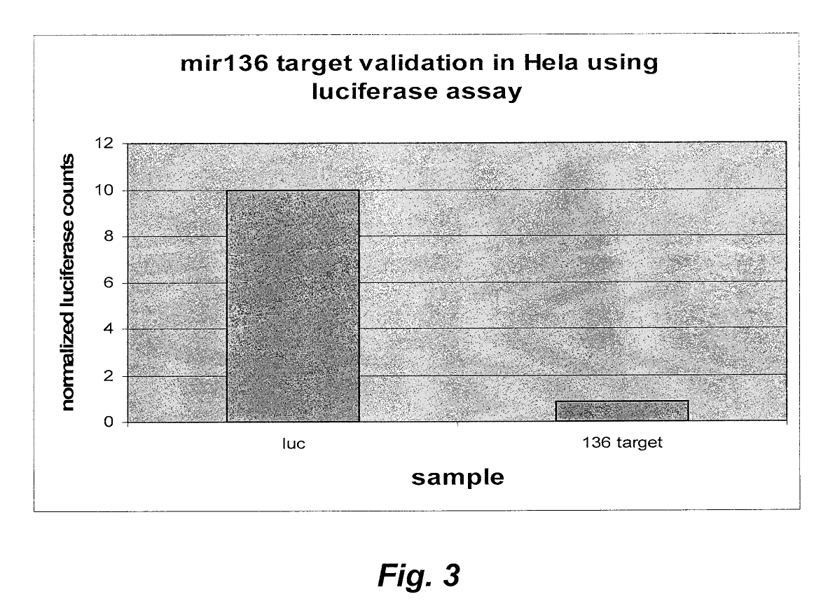

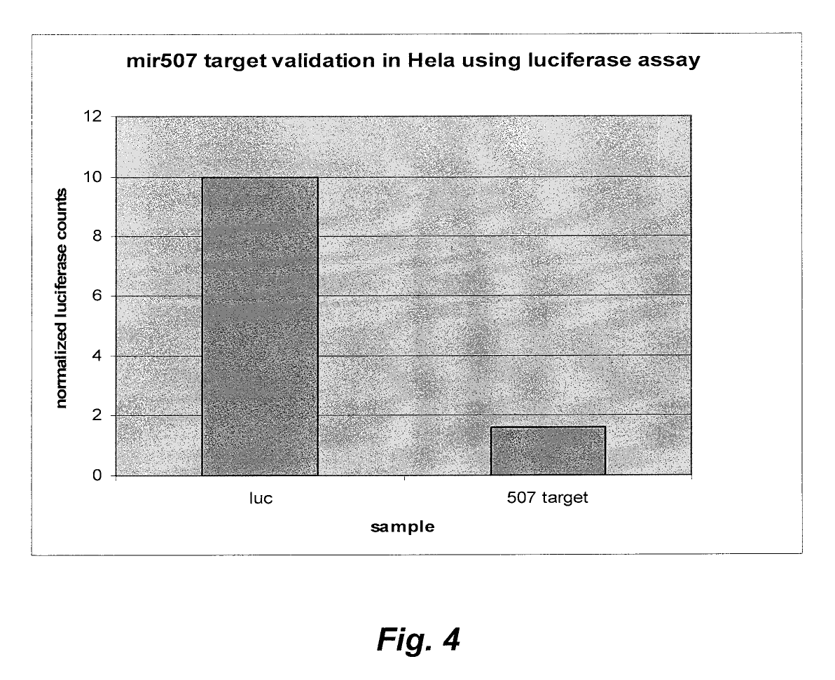

Targets for miRNA 136 and 507 within the Hemagglutinin and PolymeraseB genes showed dependence on the miRNA in the Hela cell since expression levels from the clone carrying the target region were downregulated compared to the expression from the vector without the target regions (FIG. 3 and FIG. 4).

Expression Profile Analysis of miRNA

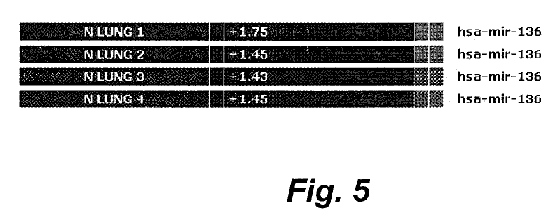

Analysis of currently available microarray based expression data revealed that human miRNAs that target SEQ ID NO 1 and 2 are expressed ubiquitously in human tissues, especially in lungs (FIG. 5).

Detection of miRNA Using Primer Extension

Detection of miRNA using Primer extension in various cell lines including the human alveolar epithelial cell line A549 revealed that the miRNA is expressed in these tissues.

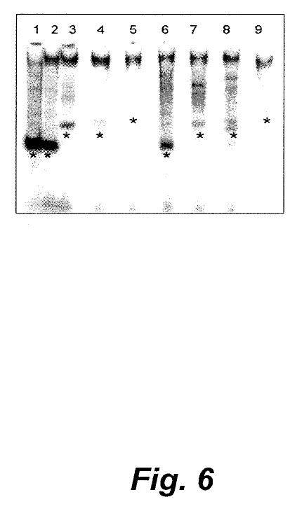

The mixture of total RNA and double autoclaved water in the ratio 1:10 was heated in a boiling water bath for 5-10 minutes followed by chilling in ice for the same duration. Subsequently, it was kept at room temperature for 10-15 minutes followed by addition of diluted dATP, dGTP and dTTP and 10 X RT buffer. 1 μl of α-P-32-dCTP was added after which RT enzyme was added. The reaction mix was then incubated at 37° C. for 30 min. The reaction was stopped by adding 2 μl 1 N NaOH and 0.5 μl 0.5 M EDTA, and the sample was incubated at 65° C. for 30 min. After 30 min 7 μl 1M Tris-HCI (pH 7.5) was added to the mixture. The samples were prepared as explained below and run on 18% polyacrylamide gel containing urea (8M). Sample Preparation: 16 M Urea was added to the samples to make the final conc. of urea to 8M. The samples were then heated at 65° C. for 5-10min, mixed with loading dye and loaded in 18% urea-containing PAGE. After running, the gel was kept in fixing solution (10% Methanol, 10% Glacial Acetic Acid) for 1 hr on a rocker. After fixing, the gel was washed with water twice, wrapped in Saran Wrap and was put for exposure. The image was scanned after overnight exposure. (FIG. 6)

Luciferase Assay

Preparation of Lysate

Lysate is prepared by suspending HeLa cells in 5X lysis buffer (CCLR, RLB or PLB) after removal of the growth medium by rinsing with PBS buffer followed by freeze thaw. The suspension is centrifuged at 12,000Xg for 15 seconds at room temperature followed by centrifugation at 4 degree centigrade for 2 minutes. The supernatant (cell lysate) is stored at minus 70 degree centigrade.

Luciferase Assay Using Luminometer

Dispense 1 φμl of the Luciferase Assay Reagent into luminometer tubes, one tube per sample. Program the luminometer to perform a 2-second measurement delay followed by a 10-second measurement read for luciferase activity. The read time may be shortened if sufficient light is produced. Add 20 μl of cell lysate to a luminometer tube containing the Luciferase Assay Reagent. Mix by pipetting 2.3 times or vortex briefly. Place the tube in the luminometer and initiate reading. (FIGS. 3-4)

Mapping of miRNA Targets:

miRNA target regions were mapped back to the reference sequence and was identified to be on segment 1 and segment 4 of H5N1 genome. Segment 1 encodes the polymerase protein PB2 and segment 4 encodes the protein haemagglutinin (HA) which are represented by SEQ ID 1 and 2 respectively.

Use of Chemically Modified miRNAs to Target HIV:

Another aspect of the invention is targeting H5N1/A virus genes using chemically modified synthetic oligomers that act as miRNAs.

The following examples are given by way of illustration of the present invention and therefore should not be construed to limit the scope of the present invention

### EXAMPLES

### Example 1

### Data Retrieval

The human microRNA mature sequences were downloaded from the database of miRNA maintained by the Sanger Center named—The miRNA Registry (microrna.sanger.ac.uk/sequences/). For querying probable targets in the H5N1/A virus genome, the inventors used the RefSeq validated H5N1/A virus reference sequence, obtained from http://ncbi.nlm.nih.

### Example 2

### Prediction of miRNA Targets in H5N1/A Virus

Four well-established microRNA target prediction softwares—miRanda, RNAhybrid, microInspector and DIANA-MicroT were used to predict targets for the human miRNAs in the H5N1/A virus reference sequence. Only those sequences were prioritized as targets which were predicted by all the four software. These short-listed H5N1/A (HIV-1) targets to human microRNAs were also found to be highly probable targets on the other prediction software. The top scoring miRNA-target pairs are tabulated in Table 1. Prior to running the RNAhybrid program, the RNAcalibrate module was used to derive the xi and theta values for calculation of Extreme Value Distribution. The xi-theta values thus obtained were included as one of the parameters while using RNAhybrid for target prediction. This minimized the base composition bias. Also, the helix parameters were set to include maximum continuous complementarity towards the 5′ end of the miRNA. It was observed that out of the several probable targets predicted by RNAhybrid, the six filtered pairs from miRanda had the lowest minimum free energy. Similar observations were made when the other two software were employed with default parameters, viz., minimum free energy of 31 20.

Validation of miRNA Targets in H5N1 Genome

The target of miRNA 136 and 507 found in the Hemagglutinin and PolymeraseB genes of H5N1 genome was further validated by experimental means. The validation was carried out in a cell culture model employing HeLa cells. Primer extension based methods described below were used to ensure that HeLa cells expressed the miRNAs being tested. A vector with the firefly luciferase gene under the control of a constitutive promoter was used to monitor the activity of the miRNA. Cultured HeLa cells were transfected with various constructs bearing reporter gene which carried testable target regions in their 3′Untranslated regions. Subsequently, the reporter gene activity was monitored using enzymatic assays. The expression level of the reporter would be expected to get downregulated if the cellular miRNA binds to the 3′UTR and results in translational block of the target gene.

Targets for miRNA 136 and 507 within the Hemagglutinin and PolymeraseB genes showed dependence on the miRNA in the Hela cell since expression levels from the clone carrying the target region were downregulated compared to the expression from the vector without the target regions.

### Example 3

### Mapping of miRNA Targets

The miRNA hsa-mir-507 [SEQ ID NO:5] targeted the PB2 gene whereas hsa-mir-136 [SEQ ID NO:6] targeted the HA gene of H5N1/A virus. The HA and PB are critical for the pathogenicity of the virus. HA is the surface glycoprotein which is involved in direct binding of the virus to the cell surface. The HA in the H5N1 subtype carries a polybasic site, cleavage at which, by cellular proteases is an essential step in establishing infection. PB2 is one of the three components of the RNP (Ribonucleoprotein) which is responsible for RNA replication and transcription. Recent evidence, from recombinant viruses generated by combinations of murine and avian viruses identified PB2 as one of the two genes associated with virulence.

### Example 4

### Comparison of Target Sequences in Related H5N 1/A Virus Strains

The variability of viral genomes can pose a problem in using RNA interference. Therefore we compared the sequence conservation at the target site amongst different H5N1/A virus strains. It was observed that the target regions were significantly conserved, using ClustalW software

### Example 5

### Expression Profile Analysis of miRNA

Microarray based expression data was retrieved from ArrayExpress database (Parkinson et al., 2005 Nucl. Ac. Res. (database issue)33:D553). The raw intensity data for each experiment was log transformed and then used for the calculation of Z scores. Z scores were calculated by subtracting the overall average gene intensity (within a single experiment) from the raw intensity data for each gene, and dividing the result by the standard deviation of all of the measured intensities, according to the formula:

Z score=(lntensity G-mean intensity G1. . . Gn)/SDG1. . .Gn.

FIG. 5 shows that hsa-miR-136 [SEQ ID NO:6] was expressed in lung tissue as well as other tissues.

Study of the expression levels of these miRNAs, in normal individuals and infected individuals who do not develop disease after prolonged periods of infection can, in future, reveal the role of human miRNA expression in accounting for differences in disease progression.

Detection of miRNA Using Primer Extension Protocol

1. In an Eppendorf tube, 1 μg of total RNA and 1 μl of primer (e.g., SEQ ID NO:3 or SEQ ID NO:4)(10 pmole/μl) were taken and the final volume was made to 10 μl using double autoclaved water.

Note: If the RNA is at a high conc. and had been stored at −20° C. for long, then warm the RNA before use.

2. The mixture was heated in a boiling water bath for 5-10 min and then chilled in ice for 5-10 min. The tube was then kept at room temp. for 10-15 min.

3. dATP, dGTP and dTTP were diluted 5 times from their stock of 2 mM each. 2 μl of each of them was then added to the reaction mixture. 10X RT Buffer was also added. Then 1 μl of α-P-32-dCTP was added to the reaction mixture and finally RT enzyme was added. The reaction mix was then incubated at 37° C. for 30min.

4. The reaction was stopped by adding 2 μl 1N NaOH and 0.5 μl 0.5M EDTA and the sample was incubated at 65° C. for 30 min. After 30 min 7 μl 1 1M Tris-HCI (pH 7.5) was added to the mixture.

5. The samples were prepared as explained below and run on 18% polyacrylamide gel containing urea (8M). 6. Sample Preparation: 16 M Urea was added to the samples to make the final conc. of urea to 8M. The samples were then heated at 65° C. for 5-10 min, mixed with loading dye and loaded in 18% urea-containing PAGE.

Note: Wash the wells properly before loading the samples.

7. After running, the gel was kept in fixing solution(10% Methanol, 10% Glacial Acetic Acid) for 1 hr on a rocker.

8. After fixing, the gel was washed with water twice, wrapped in Saran Wrap® and was put for exposure.

9. The image was scanned after overnight exposure.

Luciferase Assay Protocol

Preparation of Lysate

1. Added 4 volumes of water to 1 volume of 5X lysis buffer. Equilibrated 1X lysis buffer to room temperature before use.

2. Carefully removed the growth medium from cells to be assayed. Rinsed cells with PBS, being careful to not dislodge attached cells. Removed as much of the PBS rinse as possible.

3. Added enough 1X lysis buffer (CCLR, RLB or PLB) to cover the cells (e.g., 400 μl/60 mm culture dish, 900 μl/100 mm culture dish or 20 μl per well of a 96-well plate). While using RLB, performed a single freeze-thaw to ensure complete lysis.

4. Rocked culture dishes several times to ensure complete coverage of the cells with lysis buffer. Scraped attached cells from the dish. Transferred cells and all liquid to a microcentrifuge tube. Placed the tube on ice.

5. Vortexed the microcentrifuge tube 10.15 seconds, then centrifuged at 12,000×g for 15 seconds (at room temperature) or up to 2 minutes (at 4° C.). Transferred the supernatant to a new tube.

6. Stored the supernatant/cell lysate at 70° C.

Luciferase Assay Using Luminometer

1. Dispensed 100 μl of the Luciferase Assay Reagent into luminometer tubes, one tube per sample.

2. Programmed the luminometer to perform a 2-second measurement delay followed by a 10-second measurement read for luciferase activity. The read time may be shortened if sufficient light is produced.

Note: While using shorter assay times, validated the luminometer over that time period to ensure that readings were taken at a flat portion of the signal curve.

3. Added 20 μl of cell lysate to a luminometer tube containing the Luciferase Assay Reagent. Mixed by pipetting 2.3 times or vortexing briefly.

4. Placed the tube in the luminometer and initiated reading.

### Example 6

### Use of Chemically Modified miRNAS to Target H5N1 Targets

Another aspect of the invention is targeting H5N1 genes using chemically modified synthetic oligomers that act as miRNAs. The nucleosides of the oligomer can be modified using strategies like Locked Nucleic Acid (LNA) or 2′-O-methyl RNA (OMe) resulting in better stability and binding to the target mRNA strand, thus enabling the repression of the H5N1 proteins.

TABLE 1
TOP SCORING MIRNA-TARGET PAIRS.

| | | | |
|---|---|---|---|
| Target | 5′ ---tccaaaaagatgcaaaa 3′ | SEQ ID NO: 1 | Segment 1 (PB2) |
| Sequence | \|\|\|\|\|\|\| \|\|\|\|\|\|\| | | |
| | 3′ gtgaggtttt-ccacgtttt 5′ | SEQ ID NO: 5 | |
| hsa-mir-507 | | | |
| Target | 5′ -------tcaaaaggcaatagatggagt 3′ | SID NO: 2 | Segment 4 (HA) |
| Sequence | \|\|\|\|\|\| \|\|\| \|\|\|\|\|\|\| | | |
| | 3′ aggtagtagtttt--gtt---tacctca 5′ | SID NO: 6 | |
| hsa-mir-136 | | | |

Advantages:

Main advantage of the invention is developed miRNA is non toxic, target specific and effective therapeutics against avian flu.
Still another advantage is development of diagnostics for detection of predisposition to bird flu.
Another advantage is its use as synthetic miRNA, based on modified nucleosides, as therapeutic to prevent or inhibit the progression of disease.

Accordingly and in view of the foregoing, the herein disclosed embodiments include those directed to:

1. Targets for human microRNAs in avian flu virus strain H5N1 genome represented by SEQ ID Nos. 1 and 2.

2. Targets as in 1, for two human microRNAs (miRNAs) hsa-miR-136 and hsa-miR-507 in H5N1/A virus genome.

3. Targets as in 1, wherein of hsa-miR-507 target the nucleotide stretches of SEQ ID NO 1 in the PB2 gene.

4. Targets as in 1, wherein hsa-mir-1 36 target the nucleotide stretches of SEQ ID NO 2 in the HA gene.

5. A method for targeting avian flu virus strain H5N1 genome with human miRNAs which comprises:
(a) downloading and computationally shuffling the whole avian flu virus strain H5N1 genome sequence from publicly available databases at URL: http://www.ncbi.nlm.gov/genomes/VIRUSES/viruses.html;
(b) computationally predicting the targets for human microRNAs in the said shuffled avian flu virus strain H5N1 genome sequences using the four prediction software miRanda, RNAhybrid, MicroInspector and DianaMicroT and computing the cut off;
(c) computationally deriving consensus predictions for microRNA-target pairs, which have scores>the cutoff of step (b) for human microRNAs;
(d) mapping computationally the human microRNA targets in the avian flu virus strain H5N1 genome using the miRNA of SEQ ID NOS. 1 and 2.

6. A method as claimed in claim 5, wherein step (a) is performed using the “ShuffleSeq” program which uses a seed stretch to perform shuffling of the genome sequence.

7. A method as in 5, wherein step (b) is performed using miRNA target prediction software miRanda, RNAhybrid, MicroInspector and DianaMicroT, which are based on the experimentally derived rules of the miRNA-mRNA interaction.

8. A method as in 5, wherein step (c) is obtained by running the software miRanda on the avian flu virus strain H5N1 genome against human microRNAs and the shuffled sequence of avian flu virus strain H5N1 genome as obtained in step (a).

9. A method as in 5, wherein the parameters used for the prediction of targets are selected from sequence complementarity, minimum free energy of the duplex and continuous seed complementarity towards the 5′ end of the microRNA.

10. Use of human miRNA as prognostic biomarker for indicating progression of avian flu infection.

11. Use of the miRNAs hsa-miR-507 and hsa-miR-136 or their homologues as novel therapeutics to prevent H5N1/A virus infection or inhibit the progression of disease by microRNA mediated inhibition of protein synthesis in H5N1/A virus.

12. Targets for human microRNA in avian flu virus strain H5N1 genome and use thereof substantially as herein described with reference to the foregoing examples.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An avian flu virus strain H5N1 genomic target for a human microRNA, comprising a polynucleotide having a nucleotide sequence that is selected from SEQ ID NO:1 and SEQ ID NO:2.

2. The H5N1 genomic target of claim 1 wherein the human microRNA is selected from the group consisting of has-miR-136 [SEQ ID NO:6] and has-miR-507 [SEQ ID NO:5].

3. The H5N1 genomic target of claim 1 which is a target of the human microRNA has-miR-507 having the sequence set forth in SEQ ID NO:5, wherein the target is in a H5N1 PB2 gene and comprises the nucleotide sequence set forth in SEQ ID NO:1.

4. The H5N1 genomic target of claim 1 which is a target of the human microRNA has-miR-1 36 having the sequence set forth in SEQ ID NO:6, wherein the target is in a H5N1 HA gene and comprises the nucleotide sequence set forth in SEQ ID NO:2.

5. A method for identifying a genomic target nucleotide sequence for a human microRNA in an avian flu virus strain H5N1 genome nucleotide reference sequence, comprising:
(a) computationally shuffling the avian flu virus strain H5N1 genome nucleotide reference sequence with sequence-shuffling software to obtain one or more shuffled avian flu virus strain H5N1 genome nucleotide reference sequences;
(b) deriving a cut-off score by running one or more microRNA target prediction software programs selected from miRanda, RNAhybrid, MicroInspector and DianaMicroT, to computationally predict one or more complementary target sequences for one or a plurality of human microRNA sequences in the shuffled avian flu virus strain H5N1 genome nucleotide reference sequences of (a) to obtain for each human microRNA sequence a first value which is said cut-off score;
(c) determining a second value for each of one or more target sequences in the avian flu virus strain H5N1 genome nucleotide reference sequence that are complementary to said one or a plurality of human microRNA sequences by running one or more of the microRNA target prediction software programs selected from miRanda, RNAhybrid, MicroInspector and DianaMicroT, to computationally predict one or more complementary target sequences for the human microRNA sequences in the avian flu virus strain H5N1 genome nucleotide reference sequences to obtain therefrom said second value;
(d) selecting one or more complementary target sequences in the avian flu virus strain H5N1 genome nucleotide reference sequence from step (c) for which the second value is greater than the cut-off score of step (b) to obtain a set of consensus predicted complementary microRNA-H5N1 genome target pairs; and
(e) computationally mapping each consensus predicted microRNA-H5N1 genome target pair of (d) to the avian flu virus strain H5N1 genome nucleotide reference sequence, and therefrom identifying a genomic target nucleotide sequence for a human microRNA in the avian flu virus strain H5N1 genome nucleotide reference sequence.

6. The method of claim 5 wherein in step (e) the microRNA-H5N1 genome target pair is computationally mapped to a target sequence in the H5N1 genome that is selected from SEQ ID NO:1 and SEQ ID NO:2.

7. The method of claim 5 wherein the sequence-shuffling software in step (a) comprises an EMBOSS2 ShuffleSeq program that performs a seed stretch to computationally shuffle the avian flu virus strain H5N1 genome nucleotide reference sequence.

8. The method of claim 5 wherein step (b) comprises running miRanda, RNAhybrid, MicroInspector and DianaMicroT microRNA target prediction software programs that are based on experimentally derived rules of miRNA-mRNA interaction.

9. The method of claim 5 wherein steps (b) and (c) each comprise running the miRanda microRNA target prediction software program.

10. The method of claim 5 wherein computational prediction of targets comprises one or more of prediction of target sequence complementarity with a microRNA sequence, prediction of minimum free energy of a microRNA-H5N1 genome target pair duplex, and prediction of continuous seed complimentarity toward a 5′ end of the microRNA.

11. A method for determining progression of an avian flu infection, comprising detecting a human miRNA level wherein the miRNA is complementary to an avian flu virus strain H5N1 genome nucleotide sequence.

12. A method for preventing avian flu virus H5N1/A infection or inhibiting avian flu virus H5N1/A disease progression, comprising administering a composition comprising a microRNA that is selected from the group consisting of has-miR-507 (SEQ ID NO:5) and has-mir-136 (SEQ ID NO:6), or a homologue thereof, wherein the composition inhibits H5N1/A viral protein synthesis.

13. An avian flu virus strain H5N1 genome-derived polynucleotide that comprises a target for a human microRNA, comprising:
a genomic target nucleotide sequence for a human microRNA in an avian flu virus strain H5N1 genome that is identified according to the method of claim 5.


SEQUENCE LISTING (6 sequences)

SEQ ID NO: 1 - DNA, 17 bp, H5N1/A Virus
tccaaaaagatgcaaaa

SEQ ID NO: 2 - DNA, 21 bp, H5N1/A Virus
tcaaaaggcaatagatggagt

SEQ ID NO: 3 - DNA, 13 bp, Artificial Sequence
  Primer sequence
tccatcatcaaaa

SEQ ID NO: 4 - DNA, 16 bp, Artificial Sequence
  Primer sequence
ttcactccaaaaggtg

SEQ ID NO: 5 - DNA, 19 bp, Artificial Sequence
  Synthetic oligomer
ttttgcaccttttggagtg

SEQ ID NO: 6 - DNA, 23 bp, Artificial Sequence
  Synthetic oligomer
actccatttgttttgatgatgga